(19) United States
(12) Patent Application Publication
(10) Pub. No.: US 2012/0082989 A1
(43) Pub. Date: Apr. 5, 2012

(54) USES FOR LONG POLAR FIMBRIAE GENES OF PATHOGENIC ESCHERICHIA COLI STRAINS

(76) Inventor: Alfredo G. Torres, Friendswood, TX (US)

(21) Appl. No.: 13/138,845

(22) PCT Filed: Apr. 9, 2010
(86) PCT No.: PCT/US10/01065

§ 371 Date: Dec. 20, 2011

Related U.S. Application Data
(60) Provisional application No. 61/212,385, filed on Apr. 10, 2009.

(51) Int. Cl. C12Q 1/68; G01N 33/559; C07H 21/04

(52) U.S. Cl. 435/6.12; 435/6.15; 536/24.33; 204/456

(57) ABSTRACT

Provided are methods for identifying lpf genes in pathogenic serotypes of the Enterobacteriaceae family and for differentiating Escherichia coli (E. coli) O157:H7 strains in an isolate using primer pairs specific to lpf gene variants, particularly IpfA1 and/or lpfA 2 genes and amplicon size of the PCR product to identify prototypic Enterobacteriaceae serotypes or more specifically, to differentiate strains of E. coli serotype O157:1-17. Differentiation further requires identifying the E. coli isolate's eae gene variant type which in combination with the IpfA1 and/or lpfA2 variant identification provides unique markers. Also provided are the primer pairs and a kit comprising the same.

### CROSS-REFERENCE TO RELATED APPLICATIONS

This international application claims benefit of priority under 35 U.S.C. §119(e) of provisional application U.S. Ser. No. 61/212,385, filed Apr. 10, 2009, now abandoned, the entirety of which is hereby incorporated by reference.

### FEDERAL FUNDING LEGEND

This invention was created in part using funds from the federal government under grant AI079154-01A2 from the National Institutes of Health. Consequently, the federal government has certain rights in this invention.

### BACKGROUND OF THE INVENTION

1. Field of the Invention

The present invention relates to the fields of pathogenic microbiology and genotyping or allele typing. More specifically, the present invention relates to, inter alia, methods for using long polar fimbriae (lpf) gene types and intimin gene types as markers to differentiate pathogenic E. coli strains.

2. Description of the Related Art

During the infectious process, enterohemorrhagic Escherichia coli (EHEC) O157:H7 adheres to the intestinal epithelium where it produces Shiga toxins responsible for the hemorrhagic symptoms associated with the bloody diarrhea or during development of the hemolytic uremic syndrome. Adhesion of E. coli O157:H7 to enterocytes induces the formation of the attaching and effacing (A/E) lesion (1-2). The attaching and effacing (A/E) phenotype is mainly conferred by the Locus of Enterocyte Effacement (LEE), a pathogenicity island containing genes encoding for structural components of a type III secretion apparatus, translocator and secreted effector proteins, an adhesin (intimin) and the intimin receptor, Tir (3). The association of intimin with Tir triggers a host cell response leading to pedestal formation, and although this phenotype is best characterized in vitro, its expression correlates with the ability of the attaching and effacing organisms to colonize the intestine and cause disease in human and other animal hosts (4). Interestingly, it has been postulated that different intimin types, i.e., differences in the amino acid sequence of the intimin proteins, influence the pattern of colonization and tissue tropism in the host (5-6). Therefore, initial experimental approaches provided evidence for the existence of at least four distinct types known as intimin α, β, γ and δ (7-8). Subsequent studies have proposed that additional intimin types exist and based on differences at the nucleotide level, they have been classified as intimins ζ, η, θ, ι, κ, etc. (9-14).

While the correlation between the expression of some of the intimin types and the tissue tropism of different E. coli strains has been demonstrated experimentally using in vitro human intestinal organ cultures (5, 15-17) very little is known about other E. coli O157:H7 colonization factors, including those controlling fimbriae expression. EHEC O157:H7 contains two non-identical lpf loci homologous to the Long Polar Fimbriae (LPF) of Salmonella enterica serovar Typhimurium (18-19). Expression of the E. coli O157:H7 lpf operon 1 (lpf1) in E. coli K-12 has been linked to increased adherence to tissue-cultured cells and has been associated with the appearance of long fimbriae (18, 20). The lpf2 operon has also been linked to adherence to epithelial cells (19) and its expression in other pathogenic E. coli strains is believe to be important for development of severe diarrhea (12, 21). E. coli O157:H7 strains harboring mutations in one or both of the lpf loci have diminished colonization abilities in swine and sheep animal models (23), and also displayed an altered human intestinal tissue tropism (24). Furthermore, the role of LPF as a colonization factor associated with persistence in the intestine was elucidated using a lamb model of infection (25).

Recently, the connection was established between regulatory proteins and expression of the lpf1 loci in response to environmental cues, and found that this fimbriae is regulated by H-NS, a protein that binds to the regulatory sequence of lpfA1 and “silences” transcription, while the LEE-encoded Ler regulator binds to the regulatory sequence and inhibits the action of H-NS (26). Further, de-regulation of the lpf1 operon produced constitutive expression of the fimbriae, a phenotype which is associated to adherence and hemagglutination phenotypes in E. coli O157:H (20).

Thus, there is a continued need in the art for improved methods for differentiating E. coli O157:H7 and other pathogenic E. coli strains. More particularly, the prior art is deficient in methods for differentiating pathogenic E. coli by using allelic variants of long polar fimbriae (lpf) genes in combination with intimin types as markers for the strains. The present invention fulfills this long standing need and desire in the art.

### SUMMARY OF THE INVENTION

The present invention is directed to a method for identifying lpf genes in pathogenic serotypes of the Enterobacteriaceae family. The method comprises preparing DNA from a sample of an Enterobacteriaceae bacteria and independently amplifying the DNA with a primer pair designed for each of a specific variant region within the long polar fimbriae (lpf) gene. The size of any produced amplicon is determined such that the specific amplicon sizes produced by the specific primer pairs identify the lpf variant gene(s) in a prototypic pathogenic serotype of a group of serotypes comprising the identified variant gene(s). A representative comparison is shown in Table 1. A representative Enterobacteriaceae is an E. coli.

The present invention also is directed to a method for differentiating strains of Escherichia coli (E. coli) O157:H7. The method comprises obtaining DNA from an E. coli O157:H7 isolate, identifying the variant type of one or both of lpfA1 or lpfA2 genes in the isolate and identifying an intimin adhesin variant type from eae gene in the isolate. The identified variant types of one or both of lpfA1 or lpfA2 genes and the eae gene are matched to a known strain of E. coli O157:H7 comprising this combination, thereby differentiating the E. coli strain in the isolate. Table 4 is a chart identifying the E. coli O157:H7 strains and their lpfA1 and/or lpfA2 and eae gene variants.

The present invention is directed further to primer pairs for amplifying polymorphic regions in a long polar fimbriae (lpf) gene. The primer pairs comprise SEQ ID NOS: 1-2 (lpfA1-1), SEQ ID NOS: 3-4 (lpfA1-2), SEQ ID NOS: 5-6 (lpfA1-3), SEQ ID NOS: 7-8, (lpfA1-4) SEQ ID NOS: 9-10 (lpfA1-5), SEQ ID NOS: 11-12 (lpfA2-1), SEQ ID NOS: 13-14 (lpfA2-2), and SEQ ID NOS: 15-16 (lpfA2-3).

The present invention is directed further still to a kit comprising the primer pairs described herein. In a related invention the present invention is directed to a kit further comprising buffers and polymerases for a PCR reaction.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1B are trees based on sequence data from lpfA1 (FIG. 1A) and lpfA2 (FIG. 1B) genes. Phylogenetic positions of the 525-bp and 603-bp E. coli O157:H7 lpfA1 and lpA2 genes from strain EDL933, respectively, and the corresponding lpfA1 and lpfA2 DNA sequences from E. coli and Salmonella strains currently available in GenBank (for the Accession numbers, see Example 1). Values for each branch indicate the occurrence (%) of the branching order in 1000 bootstrapped trees. E. coli strains (serotypes) listed include: EDL933, EC4115, and Sakai (O157:H7); DEC5A (O55:H7); DEC7A (O157:H43); DEC8B (O111:H8); DEC10A (O26:H11); DEC11A (O128:H2); DEC15A (O111:H21); ECOR7 (O85:HNT); ECOR23 (O86:H43); ECOR28 (O104:NM); ECOR30 (O13:H21); ECOR33 (O7:H21); ECOR36 (O79:H25); ECOR40 (O7:NM); ECOR42 (ONT:H26); ECOR46 (O1:H6); ECOR48 (ONT:HNT); ECOR65 (ONT:H10); ECOR67 (O4:H43); O119-53 (O119:NM); E2348/69 (O127:H6); EH41 (O113:H21); O44-20 (O44); IAI1 (O8); 83/39 (O15:H-); O26:H11 (O26:H11); ED1a (O81); 789 (O78); chi7122 (O78:H9); SE11 (O152:H28); O15 (verocytotoxigenic E. coli O15); E24377A (enterotoxigenic E. coli O139:H28); 55989 (enteroaggregative E. coli). Salmonella enterica serovar listed include: P125109 (S. Enteritidis); CT02021853 (S. Dublin); SL254 (S. Newport); SL476 (S. Heidelberg); LT2 (S. Typhimurium).

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, composition or method described herein can be implemented with respect to any other device, composition or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

In one embodiment of the present invention there is provided for identifying lpf genes in pathogenic serotypes of the Enterobacteriaceae family, comprising preparing DNA from a sample of an Enterobacteriaceae bacteria; independently amplifying the DNA with a primer pair designed for each of a specific variant region within the long polar fimbriae (lpf) gene; and determining the size of any produced amplicon, wherein the specific amplicon sizes produced by the specific primer pairs identify the lpf variant gene(s) in a prototypic pathogenic serotype of a group of serotypes comprising the identified variant gene(s).

In this embodiment the size of the amplicon may be determined by gel electrophoresis. Also, in this embodiment the lpfA gene may be lpfA1 or lpfA2. In addition, the lpfA1 gene variants are lpfA1-1, lpfA1-2, lpfA1-3, lpfA1-4, or lpfA1-5 and the lpfA2 geme variants are lpfA2-1, lpfA2-2 or lpfA2-3. Furthermore, the Enterobacteriaceae bacteria may be an Escherichia, a Shigella, a Salmonella, a Citrobacter, or other diarrheagenic enteric pathogen.

In an aspect of this embodiment Enterobacteriaceae bacteria may be an E. coli and the primer pairs may be SEQ ID NOS: 1-2, SEQ ID NOS: 3-4, SEQ ID NOS: 5-6, SEQ ID NOS: 7-8, SEQ ID NOS: 9-10, SEQ ID NOS: 11-12, SEQ ID NOS: 13-14, SEQ ID NOS: 15-16.

In this aspect a 222 bp amplicon produced by the primer pair SEQ ID NO: 1-2 identifies the lpfA1-1 variant gene in the prototypic E. coli serotype O127:H6. In another aspect a 273 bp amplicon produced by the primer pair SEQ ID NO: 3-4 identifies the lpfA1-2 variant gene in the prototypic E. coli serotype O26:H11. In yet another aspect a 244 bp amplicon produced by the primer pair SEQ ID NO: 5-6 identifies the lpfA1-3 variant gene in the prototypic E. coli serotype O157:H7. In yet another aspect a 273 bp amplicon produced by the primer pair SEQ ID NO: 7-8 identifies the lpfA1-4 variant gene in the prototypic E. coli serotype ONT:H10. In yet another aspect a 250 bp amplicon produced by the primer pair SEQ ID NO: 9-10 identifies the lpfA1-5 variant gene in the prototypic E. coli serotype ONT:H26. In yet another aspect a 207 bp amplicon produced by the primer pair SEQ ID NO: 11-12 identifies the lpfA2-1 variant gene in the prototypic E. coli serotype O113:H21. In yet another aspect a 297 bp amplicon produced by the primer pair SEQ ID NO: 13-14 identifies the lpfA2-2 variant gene in the prototypic E. coli serotype O157:H7. In yet another aspect a 207 bp amplicon produced by the primer pair SEQ ID NO: 15-16 identifies the lpfA2-3 variant gene in the prototypic E. coli serotype O44.

In another embodiment of the present invention there is provided method for differentiating strains of Escherichia coli (E. coli) O157:H7, comprising obtaining DNA from an E. coli O157:H7 isolate; identifying the variant type of one or both of lpfA1 or lpfA2 genes in the isolate; identifying an intimin adhesin variant type from eae gene in the isolate; and matching the identified variant types of one or both of lpfA1 or lpfA2 genes and the eae gene to a known strain of E. coli O157:H7 comprising this combination, thereby differentiating the E. coli strain in the isolate.

In this embodiment identifying the gene variant types may comprise independently amplifying the sample DNA with primer pairs specific for the lpfA1 variant, the lpfA2 variant and the eae variant; and identifying the variant by matching a specific amplicon size produced by the specific primer pair to the variant type. As such the E. coli O157:H7 may be isolated in vitro or in vivo. Also in this embodiment the lpfA1 variant primer pairs may have the sequences shown in SEQ ID NOS: 1-2, SEQ ID NOS: 3-4, SEQ ID NOS: 5-6, SEQ ID NOS: 7-8, or SEQ ID NOS: 9-10 and the lpfA2 variant primer pairs may have the sequences shown in SEQ ID NOS: 11-12, SEQ ID NOS: 13-14, or SEQ ID NOS: 15-16. In addition eae gene variants may be γ1 (gamma), α2 (alpha), β1 (beta), θ1 (theta), ε (episilon), ε4, ζ1 (zeta), ζ3, ι1 (iota), ο (omicron), or ρ (rho). Furthermore, the differentiated E. coli O157:H7 strains are shown in Table 4.

In yet another embodiment of the present invention there is provided primer pairs for amplifying polymorphic regions in long polar fimbriae (lpf) genes. In this embodiment the primer pairs may have the sequences shown in SEQ ID NOS: 1-2, SEQ ID NOS: 3-4, SEQ ID NOS: 5-6, SEQ ID NOS: 7-8, SEQ ID NOS: 9-10, SEQ ID NOS: 11-12, SEQ ID NOS: 13-14, or SEQ ID NOS: 15-16. Also, the lpf gene may be lpfA1 and the polymorphic regions are lpfA1-1, lpfA1-2, lpfA1-3, lpfA1-4, or lpfA1-5 or the lpf gene may be lpfA2 and the polymorphic regions are lpfA2-1, lpfA2-2 or lpfA2-3.

In a related embodiment there is provided a kit for amplifying a polymorphic region of a long polar fimbriae (lpf) gene, comprising the primer pairs as described supra. Further to this related embodiment the kit may comprise the buffers and polymerases for a PCR reaction.

The Long Polar Fimbriae (Lpf) is one of few adhesive factors of enterohemorrhagic Escherichia coli O157:H7 associated with colonization of the intestine. E. coli O157:H7 strains possess two lpf loci encoding highly regulated fimbrial structures. As described herein, database analysis of the genes encoding the major fimbrial subunits demonstrated that they are present in pathogenic E. coli strains, including commensal, as well as intestinal and extra-intestinal pathogenic E. coli isolates, Salmonella strains and other Enterobacteriaceae, such as Shigella, Citrobacter, etc. The lpfA1 and lpfA2 genes are highly prevalent among LEE-positive E. coli strains associated with severe and/or epidemic disease. Further DNA sequence analysis of the lpfA1 and lpfA2 genes from different Attaching and Effacing E. coli strains resulted in the identification of several polymorphisms and the classification of the major fimbrial subunits in distinct variants.

Using collections of pathogenic E. coli isolates from Europe and Latin America, it was demonstrated that the different lpfA types are associated with the presence of specific intimin (eae) adhesin variants. Most importantly, the intimin adhesive variants are found in specific E. coli pathotypes. The present invention demonstrates that variants of the lpfA1 and lpfA2 genes are restricted to strains carrying intimin type γ, mainly EHEC O157:H7 and aEPEC O55:H7. Thus the use of these fimbrial genes as markers, in combination with the different intimin types, is useful for a specific test to identify, inter alia, the highly virulent E. coli serotype O157:H7, from other pathogenic E. coli strains.

Provided herein are methods for identifying diarrheagenic Escherichia coli (E. coli) serotypes by the long polar fimbriae (lpf) gene variants or alleles comprising the bacteria. Specifically, the method can distinguish among the lpfA1 gene variant alleles lpfA1-1, lpfA1-2, lpfA1-3, lpfA1-4, and lpfA1-5 and the lpfA2 variant alleles lpfA2-1, lpfA2-2 and lpfA2-3 alleles in these E. coli which are associated with different serotypes. Primer pairs are designed, as described in Example 1, to amplify a specific variant allele. Sequences of the primer pairs are provided in Table 1. Amplification of a sample of DNA from E. coli may be performed with a specific primer pair via PCR as is known and standard in the art. The size of the amplicon product may be determined by known and standard gel electrophoretic methods. Knowing which primer pair was utilized for amplification in combination with the size of the amplicon provides for identification of the E. coli serotype as also is shown in Table 1.

Thus, also provided are methods for differentiating among virulent isolates of Escherichia coli (E. coli) O157:H7 serotypes. The combination of the three gene markers lpfA1 and/or lpfA2 and eae are useful to perform a quick identification of the isolates. This method is useful to identify outbreak strains of specific E. coli pathotypes which occur in defined locations around the world. The strains are differentiated by the combination of lpfA1 and/or lpfA2 and eae gene variants expressed by the strains as shown in Table 4. The lpfA1 and lpfA2 variants are as described supra, The eae gene variants may be, but are not limited to, γ1 (gamma), α2 (alpha), β1 (beta), θ1 (theta), ε (episilon), ε4, ζ1 (zeta), ζ3, ι1 (iota), ο (omicron), or ρ (rho). The lpfA1, lpfA2 and eae variant types may be identified PCR as is known in the art. The specific primer pairs identified in Table 1 are used to prime an E. coli O157:H7 isolate and the amplicon produced thereby is run on a standard gel electrophoresis to determine size which corresponds to lpfA variant associated with the primer pair. The eae variants are also identified by PCR using known primers.

It is contemplated that the methods provided herein are useful to identify the lpf genes comprising other Enterobacteriaceae with genomes comprising an lpf gene, particularly lpA gene, variant alleles, such as, but not limited to, other E. coli, Shigella, and Salmonella and other enteric pathogens that cause diarrhea or other complications. Without being limited by theory, primer pairs are readily designed, as described herein for polymorphic regions of lpf genes of other Enterobacteriaceae. Correlation between the size of an amplicon(s) produced from such designed primer pair(s) and the amplicon(s) size identify the lpf variant gene(s) in a prototypic bacterial serotype of a group of serotypes comprising the identified variant gene(s) in the Enterobacteriaceae. It is contemplated further that the methods provided herein are useful to differentiate among strains of diarrheagenic Enterobacteriaceae. The specific lpf variant gene types or in combination with a marker, such as the presence of one or more other variant gene types would provide a useful means to differentiate among the various strains comprising a particular serotype of an Enterobacteriaceae bacteria.

The present invention further provides the primer pairs each of which is specific to amplify an lpf gene variant. The primer pairs and their corresponding lpf gene variant are shown in Table 1. As such, a kit is provided comprising the primer pairs and, optionally, buffers and polymerases for a PCR reaction.

The following example(s) are given for the purpose of illustrating various embodiments of the invention and are not meant to limit the present invention in any fashion.

### Example 1

### Materials and Methods

### Bacterial Strains, Plasmids, Media and Growth Conditions

Diarrheagenic and extra-intestinal pathogenic E. coli strains from the reference labs in Spain, Chile and Brazil were employed (10, 12, 28-29). The Spain collection comprised 100 strains including 18 Shiga toxin-producing E. coli (STEC), 30 enteropathogenic E. coli (EPEC), and 52 atypical enteropathogenic E. coli (aEPEC). The Chilean collection comprised 125 strains, including 64 STEC, 39 enteropathogenic E. coli, and 22 atypical enteropathogenic E. coli strains. Finally, the collection from Brazil comprised 4 enteropathogenic E. coli and 33 atypical enteropathogenic E. coli strains. For the PCR tests, enteropathogenic E. coli strain EDL933 and E. coli K-12 MG1655 were used as positive and negative controls, respectively. Strains were maintained at −80° C. and when needed, they were grown in Luria-Bertani (LB) broth (30) at 37° C.

### Recombinant DNA Techniques

Standard methods were used to perform genomic DNA isolation, PCR, and gel electrophoresis (31). Recombinant Taq polymerase enzyme (1 unit) was used in combination with 2 mM MgCl₂ and 1 μM oligonucleotide primer in each reaction. All amplifications began with a five-minute hot start at 94° C. followed by 35 cycles of denaturing at 94° C. for 30 s, annealing for 30 s in a range of 52° C.-72° C. (depending of the lpfA variant amplified), and extending at 72° C. for 30 s. In some cases, PCR reactions were performed with boiled bacterial colonies. On the basis of multiple sequence alignments, the polymorphic regions in the lpfA genes were chosen (see below), and PCR primers were derived from those regions with the help of OLIGO primer analysis software. All oligonucleotide primers are listed in Table 1.

### Phylogenetic Analysis and Gene Accession Numbers

The E. coli and Salmonella lpfA gene sequences available from public databases were analyzed using the Discovery Studio gene v.1.5 program (Accelrys). Multiple sequence alignments were performed using ClustalW with open and extended gap penalties of 10.0 and 5.0, respectively. Bootstrap subsets (1000 sets) and phylogenetic trees were generated with the neighbour-joining algorithm and the distance model used was Kimura two-parameter (32).

LpfA1 protein accession numbers: E. coli serotypes O157:H7 (EDL933, AAG58695), O157:H7 (EC4115, ACI36002), O157:H7 (Sakai, BAB37854); O55:H7 (DEC5A, BAE48422); ONT:H26 (ECOR42, BAE48423); O119:NM (O119-53, BAE48424); O127:H6 (E2348/69, CAS11346), O8 (IAI1, CAR00508); O26:H11 (BAD69589); O81 (ED1a, CAR10220); O4:H43 (ECOR67, BAE48419); O111:H21 (DEC15A, BAE48418); O111:H8 (DEC8B, BAE48417); O104:NM (ECOR28, BAE48416); O86:H43 (ECOR23, BAE48415); O128:H2 (DEC11A, BAE48420); ONT:H10 (ECOR65, BAE48421); rabbit enteropathogenic E. coli O15:H- (83/39, AAO22843); enteroaggregative E. coli 55989 (CAV00478); Salmonella enterica serovar Enteritidis (P125109, CAR35040); S. Dublin (CT02021853, ACH74212); S. Newport (SL254, ACF63868); S. Heidelberg (SL476, ACF70317); S. Typhimurium (LT2, AAL22500)

LpfA2 protein accession numbers: E. coli serotypes O157:H7 (EDL933, AAG58930), O157:H7 (EC4115, ACI39341), O157:H7 (Sakai, BAB38093); O55:H7 (DEC5A, BAE48400); O119:NM (O119-53, BAE48402); ONT:H26 (ECOR42, BAE48401); O113:H21 (EH41; AAL18161); O152:H28 (SE11; BAG79542); O78 (789; AAY18076); O78:H9 (chi7122; AAS99229); O13:H21 (ECOR30, BAE48408); O7:H21 (ECOR33, BAE48407); O26:H11 (DEC10A, BAE48410); O111:H8 (DEC8B, BAE48409); O86:H43 (ECOR23, BAE48406); O157:H43 (DEC7A, BAE48405); O104::NM (ECOR28, BAE48404); O85:HNT (ECOR7; BAE48403); ONT:HNT (ECOR48, BAE48413); O1:H6 (ECOR46, BAE48412); O7:NM (ECOR40, BAE48411); O79:H25 (ECOR36, BAE48414); O8 (IAI1; CAR00706); enterotoxigenic E. coli O139:H28 (E24377A; ABV19201); verotoxigenic E. coli O15 (AAT76975); enteroaggregative E. coli (55989, CAV00812), O44 (O44-20; BAE48399).

### Statistical Analysis

ANOVA and Pearson's chi square test were used to test associations between the clinical courses of E. coli O157:H7 infections (acute diarrhea, bloody diarrhea or HUS) and the presence of the lpfA genes.

### Example 2

### Phylogenetic trees based on LpfA1 and LpfA2 Gene Sequences of E. coli and Salmonella Strains

Previously, it has been demonstrated that the lpfA1 and lpfA2 genes are highly prevalent among LEE-positive E. coli strains, including EHEC O157:H7 strains associated with severe and/or epidemic disease (19, 33-34). Further, homologues of lpf genes have also been detected in non-O157:H7 LEE-positive E. coli strains, LEE-negative pathogenic E. coli and rabbit EPEC strains (21-22, 35-36). Therefore, a BLAST analysis was performed to identify the currently available DNA sequences in the database that display homology to the lpfA1 and lpfA2 genes of EHEC O157:H7 strain EDL933.

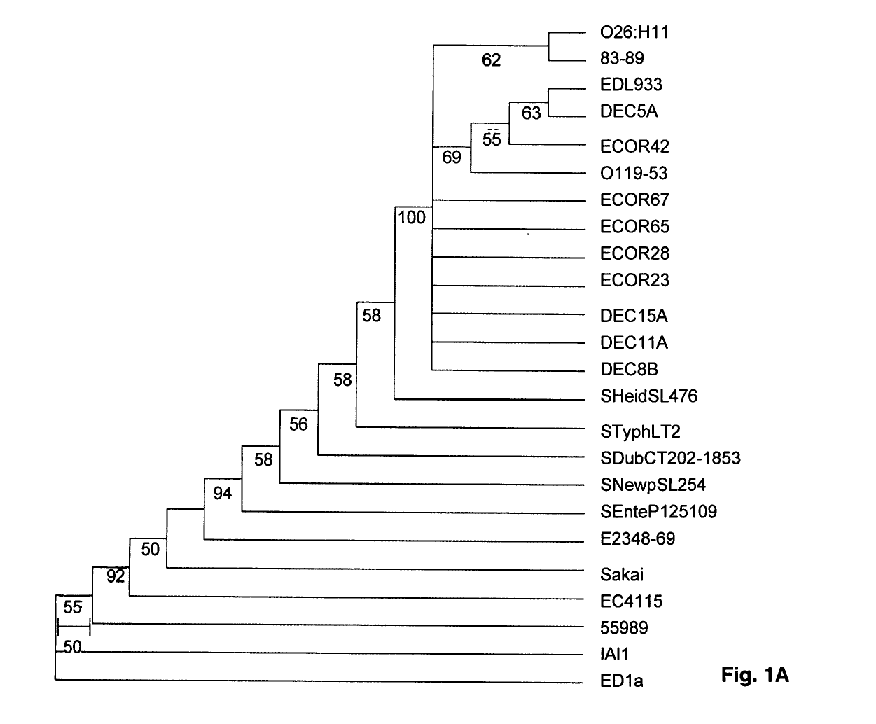

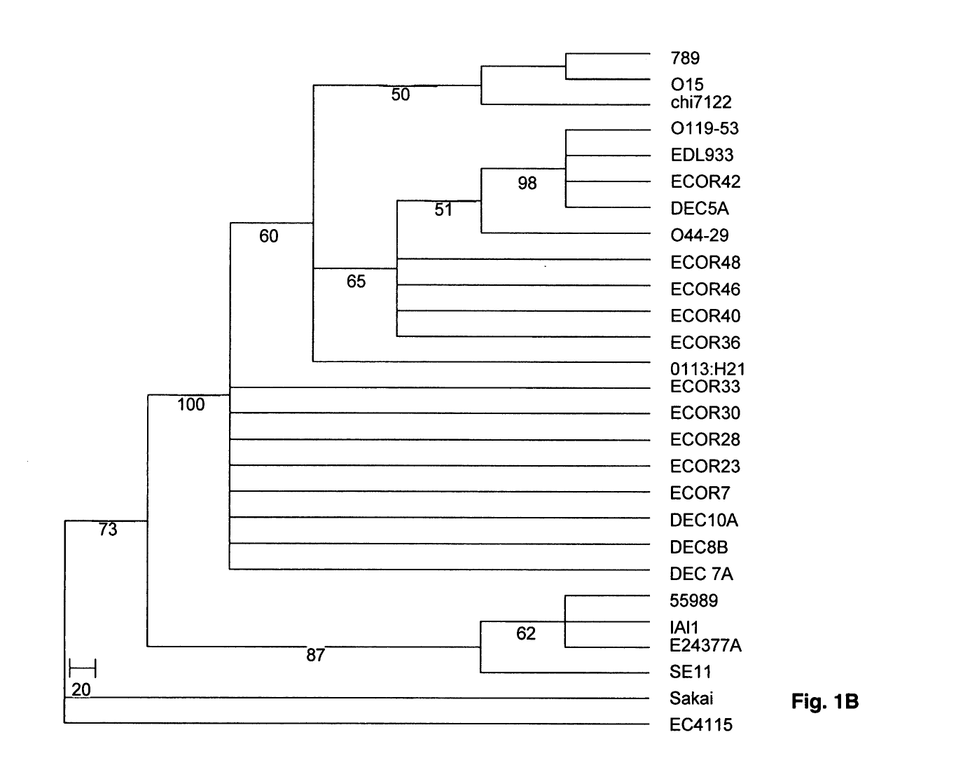

Using phylogenetic analysis of DNA sequences, distinct clades could be distinguished corresponding to the diversity of lpfA1 and lpfA2 genes (FIGS. 1A-1B). In the case of the lpfA1 gene, it was found that genes with identity ranging from 69-99% to the EDL933 lpfA1 gene were present in a range of E. coli strains including EPEC strains DEC11A, DEC5A, O119-53, and E2348/69; STEC strain DEC8B; enteroaggregative E. coli (EAEC) strains DEC15A and 55989; EHEC strains Sakai, EC4115, and O26:H11; rabbit enteropathogenic E. coli (REPEC) strain 83-39; E. coli reference collection (ECOR) strains ECOR67, ECOR65, ECOR28, ECOR23, and ECOR42; extra intestinal pathogenic E. coli (ExPEC) strain IAI1; commensal E. coli strain ED1a; and Salmonella enterica serovars Dublin (CT02021853), Newport (SL254), Enteritidis (P125109), Heidelberg (SL476) and Typhimurium (LT2).

As previously described, the EDL933 lpfA1 gene is phylogenetically related to the lpfA genes found in EPEC DEC5A, O119-53, and ECOR42 (96-99% identity) and less related (69% identity) to the lpfA1 genes found in the different serovars of Salmonella (FIG. 1A). Surprisingly, the lpfA1 genes from O157:H7 strains Sakai and EC4115 were more phylogenetically related at the nucleotide level to EPEC O127:H6 strain E2348/69 than to O157:H7 EDL933. The lpfA1 gene was also present in several strains of the ECOR reference collection and these genes shared a close phylogenetical relationship to the genes found in the other ECOR strains and DEC15A, DEC11A and DEC8B strains. Three new genome sequences recently included in the database indicated that the lpfA1 gene were also present in EAEC strain 55989, ExPEC strain IAI1 and commensal E. coli strain ED1a (FIG. 1A). Finally, the two strains of E. coli that carried the more distantly related (72-73% identity) lpfA1 genes are the REPEC 83-39 and the EHEC O26:H11 strains.

The tree analysis of the lpfA2 genes revealed a totally distinct distribution of the genes and indicated that the EDL933 lpfA2 gene is also closely related at the nucleotide level (98-99% identity) to the genes found in EPEC DEC5A, O119-53, and ECOR42 strains (FIG. 1B). Genes with homology to EDL933 lpfA2 are also found in EPEC strains O119-53 and DEC5A; STEC strains O15, DEC10A and DEC8B; EHEC strains Sakai, EC4115 and O113:H21; and members of the ECOR reference collection (ECOR42, ECOR48, ECOR46, ECOR40, ECOR36, ECOR33, ECOR30, ECOR28, ECOR23 and ECOR7). Similar to the lpfA1 tree analysis, addition of new genome sequences to the database demonstrated that genes with homology to lpfA2 are also present in other categories of pathogenic E. coli, such as EAEC strains O44-20 and 55989; ETEC strains E24377A and DEC7A; avian pathogenic E. coli (APEC) strain chi7122; ExPEC strains IAI1 and 789, and commensal E. coli strains ED1a and SE11 (FIG. 1B). A database search analysis also revealed that genes with homology to lpfA2 were also present in Shigella sonnei, S. flexneri and S. boydii.

Prevalence of Lpf1 and Lpf2 Genes in Reference Collections of Pathogenic E. coli

Because a large portion of lpfA DNA sequences available in the database belong to the pathogenic E. coli strains producing Attaching and Effacing lesions (Attaching and Effacing E. coli, AEEC), the lpfA genes might contain conserved regions useful for classifying the lpfA genes in different types (variants), and that these variants are present in specific virulent serotypes. The available DNA sequences were aligned and several conserved regions were found, allowing the lpfA1 genes to be grouped in at least 5 different types (alleles 1, 2, 3, 4 and 5), and the lpfA2 genes in 3 distinct types (alleles 1, 2 and 3).

Using these conserved regions, pairs of oligonucleotides were designed, as shown in Table 1, that specifically amplified these segments in the different lpfA types, and then determined by PCR analysis whether these lpfA variants were present in all or just in specific subsets of AEEC strains as well as E. coli strains of reference collections. As indicated in Table 1, using the diarrheagenic E. coli (DEC; strains were provided by Thomas Whittam, Michigan State University) and ECOR reference collections, as well as other prototypic AEEC strains, it was determined that the different lpfA types are present in a wide variety of serotypes and a not apparent correlation was observed between the type of lpfA1 and/or lpfA2 genes and the bacterial pathotype. Such observations has been previously reported (34); however, it is contemplated that a relationship between the lpfA type and the bacterial phylogenetic group existed.

TABLE 1

| Gene type | | | |
|---|---|---|---|
| (pre- | | | Ampli- |
| dominate | | | con |
| serotype/ | | | length |
| Primers | Sequence (5′-3′) | Position | (bp) |
| IpfA1 | | | |
| 1 (O127:H6) | | | |
| LPFA1-AF | AGTTGGTGATAAATCACCAT | 186-205 | 222 |
| | SEQ ID NO: 1 | | |
| LPFA1-AR | GTGCTGGATTCACCACTATTCATCG | 383-407 | |
| | SEQ ID NO: 2 | | |
| 2 | | | |
| (O26:1-111) | | | |
| LPFA1-B1F | AAGTCTGTATTTACTGCTATG | 169-189 | 273 |
| | SEQ ID NO: 3 | | |
| LPFA1-B1R | GAAATACAGAACGGTCTGA | 423-441 | |
| | SEQ ID NO: 4 | | |
| 3 (O157:H7) | | | |
| LPFA1-CF | GGTTGGTGACAAATCCCCG | 186-204 | 244 |
| | SEQ ID NO: 5 | | |
| LPFA1-CR1 | CGTCTGGCCTTTACTCAGA | 411-429 | |
| | SEQ ID NO: 6 | | |
| 4 (ONT:H10) | | | |
| LPFA1-B2F | AAGTCTGTGTTTACCACTACT | 64-84 | 273 |
| | SEQ ID NO: 7 | | |
| LPFA1-B2R | AAAATACAGAACAGTCTGG | 318-336 | |
| | SEQ ID NO: 8 | | |
| 5 (ONT:H26) | | | |
| LPFA1-CF | GGTTGGTGACAAATCCCCG | 81-99 | 250 |
| | SEQ ID NO: 9 | | |
| LPFA1-CR1 | GAGAACCGTCTGGCCTGTTT | 311-330 | |
| | SEQ ID NO: 10 | | |
| IpfA2 | | | |
| 1 | | | |
| (O113:H21) | | | |
| LPFA2-B1F | GGTAGTCTGGCGTCGCCACAGA | 130-151 | 207 |
| | SEQ ID NO: 11 | | |
| LPFA2-B1R | AATACGAATACCAACGCCG | 318-336 | |
| | SEQ ID NO: 12 | | |
| 2 (O157:H7) | | | |
| LPFA2-CF | CTACAGGCGGCTGATGGAACA | 61-81 | 297 |
| | SEQ ID NO: 13 | | |
| LPFA2-CR | GCTAATACCAGCGGCAGCATCGT | 335-357 | |
| | SEQ ID NO: 14 | | |
| 3 (O44) | | | |
| LPFA2-B2F | GGTAGTCTGGCGTCACCACAGC | 190-211 | 207 |
| | SEQ ID NO: 15 | | |
| LPFA2-B2R | AATACGAATACCGACACCC | 378-396 | |
| | SEQ ID NO: 16 | | |

Because it has been determined that AEEC strains possess distinct variants of intimin and some of the genes encoding these proteins are associated with specific pathotypes (37), it was determined whether there was an association between intimin (eae), the lpfA types, and the different pathotypes. Table 2 lists different lpfA types and their association with intimin types in reference collections of virulent E. coli strains. An interesting correlation emerged from this association, i.e., the lpfA1-1 variant was only present in those E. coli strains carrying the intimin genes types α1, δ/κ, η1, η2, λ, μ, and π, where EPEC O127:H6 is the representative prototype strain of that group. In the case of lpfA1-2, this gene is associated with E. coli strains carrying intimin types β1, γ2/θ, ε1, ε2. Surprisingly, the lpfA1-3 gene was only found in AEEC strains belonging to the serotype O157:H7 and in O55:H7 (both of these serotypes possess the intimin γ1 and this type of intimin is only found in EHEC O157 strains and in some of the phylogenetically related serotypes O55:H7 and O145). In contrast, no association with any intimin type was found in the strains carrying the lpfA1-4 and lpfA1-5 gene types.

TABLE 2

| | Predominant serotype strains | Association with specific |
|---|---|---|
| IpfA type | possessing the IpfA type | intimin (eae) type^{a} |
| IpfA1-1 | EPEC O127:H6 (E2348/69) | α1 (alpha), δ (delta), |
| | | κ (kappa), η1 (eta), |
| | | η2, λ (lambda), |
| | | μ (mu), π (Pi) γζ |
| IpfA1-2 | REPEC O15:H- (83/39), | β1 (beta), θ1 (theta), |
| | EPEC2 O128:H2 (DEC11a), | ε1 (epsilon), ε2 |
| | O86:H43 (ECOR23), EPEC | |
| | O128:H21 (DEC15A), | |
| | O4:H43 (ECOR67), EHEC2 | |
| | O111:H8 (DEC8B), O104:NM | |
| | (ECOR28), and O26:H11 | |
| | (AB161111) | |
| IpfA1-3 | EHEC1 O157:H7 (EDL933) | γ1 (gamma) |
| | and EHEC O55:H7 (DEC5A) | |
| IpfA1-4 | ONT:H10 (ECOR65) | NA |
| IpfA1-5 | ONT:H26 (ECOR42) and | NA |
| | O119:NM (O119-53) | |
| IpfA2-1 | EHEC O113:H21, O15, chi7122 | β1, θ1, ε2, ζ1 (zeta), |
| | (O78:K80:H9), 798-078, O85:H- | ι1B (iota), ι1C |
| | (ECOR07), O104:NM (ECOR28), | |
| | ETEC O157:H43 (DEC7A), | |
| | O86:H43 (ECOR23), O7:H21 | |
| | (ECOR33), EHEC2 O111:H8 | |
| | (DEC8B) and EHEC2 | |
| | O26:H11 (DEC10A) | |
| IpfA2-2 | EHEC1 O157:H7 (EDL933), | γ1 |
| | EHEC O55:H7 (DEC5A), | |
| | ECOR42 and O119-53 | |
| IpfA2-3 | O44 (O44-20), O1:H6 | NA |
| | (ECOR46), ONT:HNT | |
| | (ECOR48), O7:NM | |
| | (ECOR40), O79:H25 | |
| | (ECOR36) | |

^{a}NA, these types of IpfA1 or IpfA2 genes were not found in any of the intimin-positive strains analyzed from the Laboratory of Reference in E. coli (LREC), Lugo, Spain.

In the case of the lpfA2 genes, the association was not as defined as observed with the lpfA1 genes. The lpfA2-1 gene was associated with E. coli strains carrying intimin types β1, γ2/θ, ε2, ζ, ι1, and in the case of lpfA2-2, this gene is associated with E. coli strains carrying intimin types γ1, (EPEC O55:H7 and EHEC O157:H7, Table 1). Interestingly, combination of the lpfA1-3 and lpfA2-2 types was only observed in serotypes O55:H7 and O157:H7. In contrast, no association with intimin types was found in the strains carrying the lpfA2-3 gene variant. This data strongly suggests that a correlation exist between the intimin and lpfA gene variants carried by different pathogenic E. coli strains. In the case of EHEC O157:H7, because the O55:H7 clinical isolates are rarely found, the use of lpfA genes as probes in combination with the use of the intimin types could result in a specific test for the O157:H7 strains and for other pathogenic E. coli strains.

The idea of distinguishing pathogenic E. coli belonging to different pathotypes, based on sequence-based comparison of their virulence-associated genes has been demonstrated (38). In such study, 12 putative virulence genes from ExPEC strains were evaluated based on single nucleotide polymorphisms and they found that only the fimH gene (encodes for a minor component of the type 1 fimbriae) polymorphisms were able to discriminate uropathogenic E. coli from other ExPEC organisms. With those concepts in mind, a comprehensive analysis of a large collection of EPEC and STEC strains for the presence of the different intimin and lpfA gene variants was performed.

### Specific Combinations of LpfA and Intimin Gene Types are Present in STEC and EPEC Strains

To determine whether the lpfA gene types could be used as a simple, inexpensive screening test for epidemiological studies of pathogenic E. coli strains, collections of EPEC and STEC strains located in a reference lab LREC in Spain, mainly representing isolates from Europe and Brazil (Table 3) were analyzed. The identification of the different intimin and lpfA gene variants in these strains produced the following results: the lpfA1-3 and lpfA2-2 alleles were only present in strains carrying the intimin γ1 gene (STEC O157:H7, EPEC O55:H7 and two rare aEPEC isolates of serotypes O33:H7 and O163:H7). These combination of alleles are not present in other STEC strains and can be used as a discriminatory tool because other STEC strains from serotypes O26:H11, O111:H- and O111:H8 possessed the lpfA1 and lpfA2 genes, however, they carried the alleles lpfA1-2 and lpfA2-1 in combination with the intimin gene types β1 and θ1 (Table 3). In the case of the EPEC strains (this pathotype represent typical EPEC strains that possess the virulence plasmid EAF and carry the BFP fimbrial genes) the majority of the strains only possess one of the two lpfA genes. The majority of the EPEC strains analyzed possess the lpfA1-1 allele in combination with the eae type α1 (O55:H6, O127:H6), δ (O86:H34), κ (O86:H34), η1 (O125:H-), η2 (ONT:H45), and μ (O55:H- and O55:H51). In contrast, the majority of the atypical EPEC strains (lack the EAF virulence plasmid and the bfp genes) possess the lpfA1-2 and lpfA2-1 genes in combination with eae types β1 and ε2 (Table 3). Overall, these results indicated that a strong correlation exists between the intimin and the lpfA gene variants, and also suggested that the presence of both lpfA1 and lpfA2 alleles is associated with pathogenic E. coli strains, particularly with those belonging to the STEC pathotype.

TABLE 3

| | | | Country of | Intimin | IpfA1 | IpfA2 |
|---|---|---|---|---|---|---|
| LREC Number | Pathotype^{a} | Serotype | origin | (eae) type | type | type |
| IH30873A-03 | aEPEC | O51:H41 | Spain | α1 | 1 | — |
| IH52368A/03 | aEPEC | O51:H49 | Spain | α1 | 1 | — |
| FV10087 | EPEC | O55:H6 | UK | α1 | 1 | — |
| FV10088-2348III | EPEC | O127:H6 | Unknown | α1 | 1 | — |
| EPEC-21 | EPEC | O127:H- | Unknown | α1 | 1 | — |
| EPEC-23 | EPEC | O142:H6 | Unknown | α1 | 1 | — |
| IH1658-A | EPEC | O157:H45 | Spain | α1 | 1 | — |
| FV10089 | EPEC | O157:H45 | Switzerland | α1 | 1 | — |
| IH26845A-05 | aEPEC | O5:H6 | Spain | α2 | — | — |
| IH9661A-04 | aEPEC | O20:H6 | Spain | α2 | — | — |
| FV10090 | aEPEC | O125:H6 | Spain | α2 | — | — |
| FV10091 | aEPEC | O63:H33 | Spain | α2 | — | — |
| FV10092 | aEPEC | O132:H34 | Spain | α2 | — | — |
| FV10094-IH27256-03-A | STEC | O26:H11 | Spain | β1 | 2 | 1 |
| O26-7 | STEC | O26:H11 | Spain | β1 | 2 | 1 |
| O26-22 | STEC | O26:H11 | Spain | β1 | 2 | 1 |
| VTH-62 | STEC | O118:H16 | Spain | β1 | 2 | 1 |
| FV10095 | EPEC | O111:H2 | Uruguay | β1 | — | — |
| EPEC-11 | EPEC | O111:H- | Unknown | β1 | — | — |
| IH22561A-04 | EPEC | O111:H- | Spain | β1 | — | — |
| FV10096 | aEPEC | O177:H11 | Spain | β1 | 2 | 1 |
| VTB-266 | STEC | O177:H11 | Spain | β1 | 2 | 1 |
| VTB-163 | STEC | O177:H11 | Spain | β1 | 2 | 1 |
| FV12055-IH40495-06-A | aEPEC | O103:H- | Spain | β1 | 2 | 1 |
| FV12056-IH46769-07-A | aEPEC | O103:H- | Spain | β1 | 2 | 1 |
| FV11582-IH11922A07 | aEPEC | O104:H2 | Spain | β1 | 2 | — |
| FV5751 | aEPEC | O104:H2 | Brazil | β1 | 2 | — |
| FV11697-T2932-2 | aEPEC | ONT:H7 | Brazil | β1 | 2 | 1 |
| IH51463A-04 | EPEC | O88:H6 | Spain | β2 | — | — |
| FV10097 | EPEC | O119:H6 | Uruguay | β2 | — | — |
| FV10098 | aEPEC | O113:H6 | Spain | β2 | — | — |
| FV4575 | aEPEC | O139:H14 | Brazil | β2 | — | — |
| IH2056A-03 | EPEC | O167:H6 | Spain | β2 | — | — |
| FV10099 | EPEC | O167:H6 | Brazil | β2 | — | — |
| FV10101-IH34136-03-C | aEPEC | O128:H7 | Spain | β3 | — | 1 |
| FV10102-IH42584-03-A | aEPEC | O128:H- | Spain | β3 | — | — |
| FV5667 | aEPEC | O33:H7 | Brazil | γ1 | 3 | 2 |
| FV10105-IH28143-03-A | aEPEC | O55:H7 | Spain | γ1 | 3 | 2 |
| IH44336A-04 | aEPEC | O55:H7 | Spain | γ1 | 3 | 2 |
| IH5027/06A | aEPEC | O55:H7 | Spain | γ1 | 3 | 2 |
| FV5701 | aEPEC | O55:H7 | Brazil | γ1 | 3 | 2 |
| FV5665 | aEPEC | O55:H7 | Brazil | γ1 | 3 | 2 |
| O157-847 | STEC | O157:H7/SF− | Spain | γ1 | 3 | 2 |
| O157-881 | STEC | O157:H7/SF− | Spain | γ1 | 3 | 2 |
| FV10103 | STEC | O157:H7/SF− | Spain | γ1 | 3 | 2 |
| FV10104-EDL933 | STEC | O157:H7 | Canada | γ1 | 3 | 2 |
| FV10108-FV5570 | STEC | O157:H-/SF+ | Germany | γ1 | 3 | 2 |
| FV10107-FV5569 | STEC | O157:H-/SF+ | Germany | γ1 | 3 | 2 |
| FV5668 | aEPEC | O163:H7 | Brazil | γ1 | 3 | 2 |
| IH7548-05A | aEPEC | ONT:H- | Spain | γ1 | 5 | — |
| IH15752-07A | aEPEC | O2 | Spain | γ1 | 5 | — |
| FV10106 | aEPEC | O145:H28 | Spain | γ1 | 5 | — |
| IH11218-04A | aEPEC | O145:H28 | Spain | γ1 | — | — |
| IH38354A/05 | STEC | O145:H- | Spain | γ1 | 5 | — |
| IH34365-05A | aEPEC | O145:H- | Spain | γ1 | — | 3 |
| IH10248A-05 | aEPEC | O2:H40 | Spain | θ1 | — | — |
| FV11585-IH8663A06 | aEPEC | O2:H49 | Spain | θ1 | — | — |
| FV10109 | STEC | O111:H- | Spain | θ1 | 2 | 1 |
| FV10110 | STEC | O111:H8 | Germany | θ1 | 2 | 1 |
| IH45218A-06 | aEPEC | O111:H25 | Spain | θ1 | — | — |
| FV10111 | EPEC | O127:H40 | Uruguay | θ1 | — | — |
| IH5098A-07 | EPEC | O131:H46 | Spain | θ1 | — | 1 |
| FV11695-T1871-1 | aEPEC | O34:H- | Brazil | θ2 | 2 | 1 |
| IH25556A-03 | EPEC | O49:H10 | Spain | κ | — | — |
| FV10114 | EPEC | O86:H34 | Brazil | δ | 1 | — |
| FV10112 | EPEC | O86:H34 | Unknown | κ | 1 | — |
| IH48480A-06 | EPEC | O88:H- | Spain | κ | — | — |
| FV10113 | EPEC | O118:H5 | Germany | κ | — | — |
| FV5713 | aEPEC | O1:H45 | Brazil | ε1 | — | — |
| FV5718 | aEPEC | O21:H38 | Brazil | ε1 | — | 1 |
| FV10116 | aEPEC | O26:H- | Brazil | ε1 | — | — |
| FV5715 | aEPEC | O80:H26 | Brazil | ε1 | — | — |
| FV10115 | STEC | O103:H2 | Spain | ε1 | 2 | — |
| FV5676 | EPEC | O111:H40 | Brazil | ε1 | — | — |
| FV10117 | aEPEC | O157:H16 | Spain | ε1 | — | — |
| FV10118 | aEPEC | O6:H19 | Spain | ε2 | 2 | 1 |
| FV10119 | aEPEC | O103:H19 | Brazil | ε2 | — | — |
| FV10120 | aEPEC | O123:H19 | Spain | ε2 | 2 | 1 |
| FV12050-IH9456-07-A | EPEC | O88:H25 | Spain | ε2 | 2 | 1 |
| FV12051-IH37508-06-A | EPEC | O88:H25 | Spain | ε2 | 2 | 1 |
| FV11698-73382-9 | aEPEC | O109:H9 | Brazil | ε2 | — | 1 |
| FV11680-T2332-7 | aEPEC | O123:H- | Brazil | ε2 | — | 1 |
| FV11681-T1482-11 | aEPEC | O123:H- | Brazil | ε2 | — | 1 |
| FV11678-T0811-4 | aEPEC | ONT:H- | Brazil | ε2 | — | 1 |
| FV10121-IH31923-03-A | aEPEC | O181:H- | Spain | ε3 | — | — |
| FV10123-IH37159-03-A | EPEC | O109:H- | Spain | ε4 | — | — |
| FV10142 | STEC | O80:H- | Spain | ε5/ζ | — | — |
| FV10143 | aEPEC | O80:H2 | Slovakia | ε5/ζ | — | — |
| FV10144 | aEPEC | O157:H- | Spain | ε5/ζ | — | — |
| FV10124 | STEC | O156:H- | Spain | ζ1 | — | 1 |
| FV10125 | aEPEC | O156:H- | Spain | ζ1 | — | 1 |
| FV11212-E110019 | aEPEC | O111:H9 | Finland | ζ2 | — | 1 |
| FV11584-IH46488A07 | EPEC | O111:H- | Spain | ζ2 | — | 1 |
| FV11677-T2381-8 | aEPEC | O154:H9 | Brazil | ζ2 | — | 1 |
| FV11682-T2232-6 | aEPEC | O49:H- | Brazil | ζ2 | — | 1 |
| FV10126 | aEPEC | O85:H31 | Spain | ζ3 | — | — |
| FV10127 | EPEC | O125:H- | Burundi | η1 | 1 | — |
| FV10128-IH53199-03-A | aEPEC | ONT:H45 | Spain | η2 | — | — |
| FV10129 | EPEC | ONT:H45 | Switzerland | η2 | 1 | — |
| FV10130 | aEPEC | O145:H4 | Germany | ι1A | — | — |
| FV5750 | aEPEC | O145:H2 | Brazil | ι1A | — | — |
| FV11583-IH47502A07 | aEPEC | O2:H49 | Spain | ι1A | — | — |
| FV10131 | EPEC | O153:H8 | Spain | ι1B | — | 1 |
| FV11679-T3281-6 | aEPEC | O85:H- | Brazil | ι1B/C | — | 1 |
| FV11868-IH40760A/06 | EPEC | O55:H8 | Spain | ι1C | — | 1 |
| FV10132 | EPEC | O119:H8 | Switzerland | ι1C | — | 1 |
| FV10133 | aEPEC | ONT:H45 | Spain | ι2 | — | — |
| FV11873-IH39717A/03 | aEPEC | O101:H- | Spain | ι2 | — | — |
| FV11874-LORENY217.2 | aEPEC | O101:H- | Brazil | ι2 | — | — |
| FV10134 | aEPEC | O34:H- | Brazil | λ | — | — |
| FV10135 | aEPEC | O33:HNT | Brazil | λ | 1 | — |
| FV10136 | aEPEC | O101:H33 | Uruguay | λ | — | — |
| FV5737 | aEPEC | O101:H33 | Brazil | λ | — | — |
| FV5752 | aEPEC | O124:H11 | Brazil | λ | 1 | — |
| FV10137 | EPEC | O55:H- | Uruguay | μ | 1 | — |
| FV10138 | EPEC | O55:H51 | Uruguay | μ | 1 | — |
| LR-88-1 | EPEC | O55:H51 | Brazil | μ | 1 | — |
| FV10139 | EPEC | O55:H- | Uruguay | μ | 1 | — |
| FV10140 | aEPEC | O10:H- | Spain | ν | — | — |
| FV10141 | aEPEC | ONT:H- | Spain | ν | — | — |
| FV10145 | aEPEC | O129:H- | Spain | ο | — | — |
| FV10146 | aEPEC | O84:H- | Spain | ο | — | — |
| FV12052-IH28343-03-A | aEPEC | O84:H- | Spain | ο | — | — |
| FV12053-IH36299-04-A | aEPEC | O105:H4 | Spain | ο | 2 | 1 |
| IH21822A | aEPEC | ONT:H- | Spain | ο | — | — |
| FV10430-T1551-2 | aEPEC | ONT:H- | Brazil | ο | — | — |
| FV10147 | aEPEC | O14:H5 | Brazil | π | 1 | — |
| FV10148 | aEPEC | ONT | Brazil | π | 1 | — |
| FV5724 | aEPEC | ONT:H5 | Brazil | π | 1 | — |
| FV10149 | aEPEC | O149:H- | Spain | ρ | — | — |
| FV10150 | aEPEC | O149:H- | Spain | ρ | — | — |
| FV10151 | aEPEC | O180:H- | Spain | ρ | — | — |
| FV10152 | aEPEC | O86:H- | UK | σ | — | — |
| FV10153 | aEPEC | O86:H- | UK | σ | — | — |
| FV10425-T0621-6 | aEPEC | ONT:H- | Brazil | σ | — | — |
| FV11772A-T4281-7 | aEPEC | O104:H- | Brazil | τ | — | — |
| FV11696-T1632-7 | aEPEC | O26:H- | Brazil | ν | — | — |

^{a}Pathotypes were established based on the presence of virulence factors: EPEC was positive for the bfpA gene and the presence of the EAF plasmid; atypical EPEC (aEPEC) was negative for bfpA and EAF; Shiga toxin-producing E. coli (STEC) carry the stx gene.
/SF− sorbitol fermenting negative; /SF+ sorbitol-fermenting positive.

A collection of strains isolated predominantly in Chile (collection located at the CVD, Chile) and with no relationship to the other reference strains were independently analyzed. Sixty two STEC O157:H7 strains were evaluated for the presence of the lpfA and intimin genes. As shown in Table 4, there was a complete correlation (100%) between the EHEC serotype O157:H7 strains and the types of intimin (eae) and lpfA gene variants. All 62 strains possessed the intimin γ1 gene and they all carried the lpfA1-3 and lpfA2-2 gene combination, strongly supporting the idea that these three genes were reliable markers to identify this highly virulent serotype.

Then, whether a correlation existed between the presence of these genes and the pathotypes in a collection of EPEC strains was determined. Table 4 shows the correlation of lpfA types with EHEC and EPEC serotypes in a collection of strains from the CVD, Chile. Different types of lpfA1 and lpfA2 genes were found in combination with intimin gene variants. However, some trends were evident: (a) the majority of lpfA genes belong to the lpfA1-2 and/or lpfA2-1 variants. (b) The strains possessing lpfA1-2 and lpfA2-1 type genes also carried the intimin gene types β1, θ1, ε, ο, and γ1. (c) The majority of the Chilean EPEC strains, whether they belong to the typical or atypical EPEC pathotype, possessed both lpfA1 and lpfA2 genes or they lack both of these genes. Interestingly, novel trends also emerged from this analysis, e.g., strains from serotype O145:H8 carrying unique combination of lpfA genes (lpfA1-5 with lpfA2-1) were identified.

TABLE 4

| Strain ref | | | Intimin | IpfA1 | IpfA2 |
|---|---|---|---|---|---|
| number^{a} | Pathotype^{b} | Serotype^{c} | (eae) type^{c} | type | type |
| O157:H7 isolates | STEC | O157:H7 | γ1 | 3 | 2 |
| Di-304 | aEPEC | O63:H6 | α2 | — | — |
| PH-78 | aEPEC | O125 | α2 | — | — |
| Di-12 | aEPEC | O15:H25 | β1 | — | 1 |
| Di-126 | aEPEC | O20:H7 | β1 | 2 | 1 |
| Di-140 | aEPEC | O20:H7 | β1 | 2 | 1 |
| Di-91 | aEPEC | O26:H- | β1 | 2 | 1 |
| O26-187 | STEC | O26:H11 | β1 | 2 | 1 |
| O26-215 | STEC | O26:H11 | β1 | 2 | 1 |
| J-80 | aEPEC | O119:H- | β1 | 2 | 1 |
| FV10037 | aEPEC | O153:H7 | β1 | 2 | 1 |
| Di-358 | aEPEC | O137:H6 | β1 | — | 1 |
| Di-178 | EPEC | ND | β1 | — | — |
| Di-210 | EPEC | O98:HNT | β1 | 2 | 1 |
| Di-282 | EPEC | O158 | β1 | — | 1 |
| Di-305 | EPEC | O119 | β1 | 2 | — |
| Di-333 | EPEC | ND | β1 | 2 | — |
| J-11 | EPEC | O126 | β1 | 2 | 1 |
| J-145 | aEPEC | ND | β1 | 2 | — |
| J-215 | EPEC | ND | β1 | — | — |
| J-275 | EPEC | ND | β1 | — | — |
| J-294 | EPEC | ND | β1 | 5 | — |
| FV9988 | EPEC | O145:H8 | γ1 | 5 | — |
| Di-141 | aEPEC | O145:H8 | γ1 | 5 | 1 |
| J-104 | EPEC | ND | γ1 | 2 | 1 |
| FV9965 | EPEC | O23:H8 | θ1 | 2 | 1 |
| FV9986 | aEPEC | O23:H8 | θ1 | 2 | 1 |
| Di-4 | aEPEC | O23:H8 | θ1 | 2 | 1 |
| J-9 | EPEC | O23:H8 | θ1 | 2 | 1 |
| FV9967 | aEPEC | O55:H40 | θ1 | — | — |
| FV10039 | EPEC | O111:H25 | θ1 | — | — |
| J-97 | aEPEC | ONT:H40 | θ1 | — | — |
| J-98 | aEPEC | O55 | θ1 | — | — |
| J-151 | EPEC | ND | θ1 | — | — |
| Di-174 | EPEC | ONT:H19 | ε | 2 | 1 |
| Di-219 | EPEC | ND | ε | 2 | 1 |
| J-168 | EPEC | ND | ε | — | — |
| PH-11 | EPEC | ND | ε | — | — |
| FV9968 | EPEC | O109:H- | ε4 | — | — |
| Di-45 | EPEC | ONT:H1,H12 | ζ1 | — | — |
| Di-224 | EPEC | ND | ζ1 | — | — |
| PH-92 | EPEC | O1:H1,H12 | ζ1 | — | 3 |
| FV9970 | EPEC | O1:H1,H12 | ζ3 | — | — |
| FV9991 | EPEC | O1:H1,H12 | ζ3 | — | 3 |
| Di-101 | EPEC | O86:H8 | ι1 | — | 1 |
| Di-307 | aEPEC | O145:H34 | ι1 | 2 | — |
| FV9969 | aEPEC | ONT:H4 | ο | 2 | 1 |
| J-222 | aEPEC | ONT:H4 | ο | 2 | 1 |
| FV9984 | aEPEC | O76:H2 | ρ | — | — |
| Di-124 | aEPEC | O76:H2 | ρ | — | — |
| Di-192 | aEPEC | O145:H34 | NT | — | — |
| J-70 | EPEC | ND | NT | — | — |
| J-206 | EPEC | ND | NT | 1 | — |
| J-280 | EPEC | ND | NT | 5 | — |
| J-303 | EPEC | ND | NT | — | — |
| J-349 | EPEC | ND | NT | — | — |
| J-351 | EPEC | ND | NT | — | — |
| J-94 | EPEC | ND | NT | — | 3 |
| J-281 | EPEC | ND | NT | — | — |
| PH-32 | EPEC | ND | NT | — | 3 |
| PH-74 | EPEC | ND | NT | 2 | 3 |
| PH-112 | EPEC | ND | NT | — | — |
| PH-120 | EPEC | ND | NT | 2 | 1 |
| S-28 | EPEC | ND | NT | — | — |

^{a}Center for Vaccine Development, Santiago, Chile.
^{b}STEC, Shiga toxin-producing E. coli; EPEC, enteropathogenic E. coli; aEPEC, atypical EPEC.
ND: not determined
NT: not typeable

Finally, the association between the presence of the lpfA gene and the type of disease produced by the different pathotypes isolated in Chile (acute diarrhea, bloody diarrhea or HUS) was determined. The statistical analysis indicated that a strong association existed between the lpfA gene and E. coli strains carrying the stx genes (STEC strains). Further, the lpfA (p=0.0058) and stx2 (p=0.0014) genes were significantly associated with HUS. Neither lpfA nor stx were significantly associated with acute diarrhea or bloody diarrhea. Therefore, it was concluded that lpfA and stx2 are STEC virulence factors that showed strong association with the HUS pathology that can result from a STEC infection.

The complete elucidation of the evolutionary history of the lpf gene clusters in diarrheagenic E. coli strains, as well as ExPEC, commensal E. coli, Shigella and Salmonella strains, is being performed. This analysis includes mapping the chromosomal location of the lpf gene clusters (in EHEC O157:H7, lpfA1 is linked to the O-island 141 [OI-141], while lpfA2 is located in OI-154), and characterization of the other open reading frames within the lpf operons, because studies with other firmbriae gene clusters have suggested that different regions within an operon have diverse evolutionary histories (39). But what is now evident is that acquisition of different lpf gene clusters in specific lineages of E. coli might be contributing to the emergence of highly virulent strains derived from commensal organisms, which also possess unique lpf variants.

The following references are cited herein.
1. Kaper, et al., 2004. Nat Rev Microbiol 2:123-140.
2. Torres, et al., 2005 Infect Immun 73:18-29.
3. Torres, A. G., and J. B. Kaper. 2001. PAIs of intestinal E. coli., p. 31-48. In J. Hacker and J. B. Kaper (ed.), Pathogenicity Islands (PAIs) and the Evolution of Pathogenic Microbes., Vol. 1. Springer-Verlag Berlin Heidelberg New York, Berlin Heidelberg.
4. Nataro, J. P., and J. B. Kaper. 1998 Clin Microbiol Rev 11:142-201.
5. Fitzhenry, et al., 2002 Gut 50:180-185.
6. Phillips, A. D., and G. Frankel. 2000 J Infect Dis 181:1496-1500.
7. Adu-Bobie et al. 1998, J Clin Microbiol 36:662-668.
8. Agin, T. S., and M. K. Wolf. 1997 Infect Immun 65:320-326.
9. Blanco, et al., 2004. J Clin Microbiol 42:645-651.
10. Blanco, et al., 2005. BMC Microbiol 5:23.
11. Jores et al., 2003 Exp Biol Med (Maywood) 228:370-376.
12. Mora, et al., 2007 BMC Microbiol 7:13.
13. Tarr, et al., 2002 J Bacteriol 184:479-487.
14. Zhang, et al., 2002 J Clin Microbiol 40:4486-4492.
15. Cantey, J. R., and L. R. Inman. 1981 Infect Dis 143:440-446.
16. Fitzhenry, et al., 2003 FEMS Microb Lett 218:311-316.
17. Phillips, et al., 2000 Gut 47:377-381.
18. Torres, et al., 2002 Infect Immun 70:5416-5427.
19. Torres, et al., 2004 FEMS Microbiol Lett 238:333-344.
20. Torres, et al., 2008 Infect Immun 76:5062-5071.
21. Doughty, et al., 2002 Infect Immun 70:6761-6769.
22. Osek, et al., 2003 Vet Microbiol 96:259-266.
23. Jordan, et al., 2004 Infect Immun 72:6168-6171.
24. Fitzhenry, et al., 2006 Int J Med Microbiol 296:547-552.
25. Torres, et al., 2007 Int J Med Microbiol 297:177-185.
26. Torres, et al., 2007 J Bacteriol 189:5916-5928.
27. Moreira, et al., 2008 J Clin Microbiol 46:1462-1465.
28. Oliveira, et al., 2008 Int J Food Microbiol 127:139-146.
29. Vidal, et al., 2007 Epidemiol Infect 135:688-694.
30. Maniatis, et al., 1982. Molecular cloning: a laboratory manual. Cold Spring Harbor Laboratory Press, N.Y.
31. Sambrook, et al., 1989. Molecular Cloning: A Laboratory Manual. Cold Spring Harbor Laboratory Press, Cold Spring Harbor, N.Y.
32. Kimura, M. 1980 J Mol Evol 16:111-120.
33. Szalo, et al., 2002 Res Microbiol 153:653-658.
34. Toma, et al., 2006 Res Microbiol 157:153-161.
35. Newton, et al., 2004 Infect Immun 72:1230-1239.
36. Toma, et al., 2004 J Clin Microbiol 42:4937-4946.
37. Reid, et al., 1999 J Clin Microbiol 37:2719-2722.
38. Tartof, et al., 2007 J Med Microbiol 56:1363-1369.
39. Boyd, E. F., and D. L. Hartl. 1999. J Bacteriol 181:1301-1308.

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

1. A method for identifying lpf genes in pathogenic serotypes of the Enterobacteriaceae family, comprising:
preparing DNA from a sample of an Enterobacteriaceae bacteria;
independently amplifying the DNA with a primer pair designed for each of a specific variant region within the long polar fimbriae (lpf) gene; and
determining the size of any produced amplicon, wherein the specific amplicon sizes produced by the specific primer pairs identify the lpf variant gene(s) in a prototypic pathogenic serotype of a group of serotypes comprising the identified variant gene(s).

2. The method of claim 1, wherein the size of the amplicon is determined by gel electrophoresis.

3. The method of claim 1, wherein the Enterobacteriaceae bacteria is an Escherichia, a Shigella, a Salmonella, a Citrobacter, or other diarrheagenic enteric pathogen.

4. The method of claim 1, wherein the lpf gene is lpfA1 or lpfA2.

5. The method of claim 1, wherein the lpfA1 gene variants are lpfA1-1, lpfA1-2, lpfA1-3, lpfA1-4, or lpfA1-5 and the lpfA2 gene variants are lpfA2-1, lpfA2-2 or lpfA2-3.

6. The method of claim 1, wherein the Enterobacteriaceae bacteria is an E. coli and the primer pairs are SEQ ID NOS: 1-2, SEQ ID NOS: 3-4, SEQ ID NOS: 5-6, SEQ ID NOS: 7-8, SEQ ID NOS: 9-10, SEQ ID NOS: 11-12, SEQ ID NOS: 13-14, SEQ ID NOS: 15-16.

7. The method of claim 6, wherein a 222 bp amplicon produced by the primer pair SEQ ID NO: 1-2 identifies the lpfA1-1 variant gene in the prototypic E. coli serotype O127:H6.

8. The method of claim 6, wherein a 273 bp amplicon produced by the primer pair SEQ ID NO: 3-4 identifies the lpfA1-2 variant gene in the prototypic E. coli serotype O26:H11.

9. The method of claim 6, wherein a 244 bp amplicon produced by the primer pair SEQ ID NO: 5-6 identifies the lpfA1-3 variant gene in the prototypic E. coli serotype O157:H7.

10. The method of claim 6, wherein a 273 bp amplicon produced by the primer pair SEQ ID NO: 7-8 identifies the lpfA1-4 variant gene in the prototypic E. coli serotype ONT:H10.

11. The method of claim 6, wherein a 250 bp amplicon produced by the primer pair SEQ ID NO: 9-10 identifies the lpfA1-5 variant gene in the prototypic E. coli serotype ONT:H26.

12. The method of claim 6, wherein a 207 bp amplicon produced by the primer pair SEQ ID NO: 11-12 identifies the lpfA2-1 variant gene in the prototypic E. coli serotype O113:H21.

13. The method of claim 6, wherein a 297 bp amplicon produced by the primer pair SEQ ID NO: 13-14 identifies the lpfA2-2 variant gene in the prototypic E. coli serotype O157:H7.

14. The method of claim 6, wherein a 207 bp amplicon produced by the primer pair SEQ ID NO: 15-16 identifies the lpfA2-3 variant gene in the prototypic E. coli serotype O44.

15. A method for differentiating strains of Escherichia coli (E. coli), comprising:
obtaining DNA from an E. coli isolate;
identifying the variant type of one or both of lpfA1 or lpfA2 genes in the isolate;
identifying an intimin adhesin variant type from eae gene in the isolate; and
matching the identified variant types of one or both of lpfA1 or lpfA2 genes and the eae gene to a known strain of E. coli comprising this combination, thereby differentiating the E. coli strain in the isolate.

16. The method of claim 15. wherein identifying the gene variant types comprises,
independently amplifying the sample DNA with primer pairs specific for the lpfA1 variant, the lpfA2 variant and the eae variant; and
identifying the variant by matching a specific amplicon size produced by the specific primer pair to the variant type.

17. The method of claim 16, wherein the lpfA1 variant primer pairs have the sequences shown in SEQ ID NOS: 1-2, SEQ ID NOS: 3-4, SEQ ID NOS: 5-6, SEQ ID NOS: 7-8, or SEQ ID NOS: 9-10.

18. The method of claim 16, wherein the lpfA2 variant primer pairs have the sequences shown in SEQ ID NOS: 11-12, SEQ ID NOS: 13-14, or SEQ ID NOS: 15-16.

19. The method of claim 15, wherein the lpfA1 gene variants are lpfA1-1, lpfA1-2, lpfA1-3, lpfA1-4, or lpfA1-5 and the lpfA2 gene variants are lpfA2-1, lpfA2-2 or lpfA2-3.

20. The method of claim 15, wherein the eae gene variants are γ1 (gamma), α2 (alpha), β1 (beta), θ1 (theta), ε (epsilon), ε4, ζ1 (zeta), ζ3, ι1 (iota), ο (omicron), or ρ (rho).

21. The method of claim 15, wherein the E. coli serotype is O157:H7; wherein the differentiated strains are shown in Table 4.

22. The method of claim 15, wherein the E. coli was isolated in vitro or in vivo.

23. Primer pairs for amplifying polymorphic regions in a long polar fimbriae (lpf) gene.

24. The primer pairs of claim 23, wherein the primer pairs have the sequences shown in SEQ ID NOS: 1-2, SEQ ID NOS: 3-4, SEQ ID NOS: 5-6, SEQ ID NOS: 7-8, SEQ ID NOS: 9-10, SEQ ID NOS: 11-12, SEQ ID NOS: 13-14, or SEQ ID NOS: 15-16.

25. The primer pairs of claim 23, wherein the lpf gene is lpfA1 or lpfA2.

26. The primer pairs of claim 25, wherein the gene is lpfA1 and the polymorphic regions are lpfA1-1, lpfA1-2, lpfA1-3, lpfA1-4, or lpfA1-5.

27. The primer pairs of claim 25, wherein the gene is lpfA2 and the polymorphic regions are lpfA2-1, lpfA2-2, or lpfA2-3.

28. A kit for amplifying a polymorphic region of a long polar fimbriae (lpf) gene, comprising:
the primer pairs of claim 23.

29. The kit of claim 28, further comprising:
buffers and polymerases for a PCR reaction.


SEQUENCE LISTING (16 sequences)

SEQ ID NO: 1 - DNA, 20 bp, artificial sequence
  forward primer LPFA1-AF for lpfA1 gene in E. coli O127H6
agttggtgataaatcaccat

SEQ ID NO: 2 - DNA, 25 bp, artificial sequence
  reverse primer LPFA1-AR for lpfA1 gene in E. coli O127H6
gtgctggattcaccactattcatcg

SEQ ID NO: 3 - DNA, 21 bp, artificial sequence
  forward primer LPFA1-B1F for lpfA1 gene in E. coli O26H11
aagtctgtatttactgctatg

SEQ ID NO: 4 - DNA, 19 bp, artificial sequence
  reverse primer LPFA1-B1R for lpfA1 gene in E. coli O26H11
gaaatacagaacggtctga

SEQ ID NO: 5 - DNA, 19 bp, artificial sequence
  forward primer LPFA1-CF for lpfA1 gene in E. coli O157H7
ggttggtgacaaatccccg

SEQ ID NO: 6 - DNA, 19 bp, artificial sequence
  reverse primer LPFA1-CR1 for lpfA1 gene in E. coli O157H7
cgtctggcctttactcaga

SEQ ID NO: 7 - DNA, 21 bp, artificial sequence
  forward primer LPFA1-B2F for lpfA1 gene in E. coli ONTH10
aagtctgtgtttaccactact

SEQ ID NO: 8 - DNA, 19 bp, artificial sequence
  reverse primer LPFA1-B2R for lpfA1 gene in E. coli ONTH10
aaaatacagaacagtctgg

SEQ ID NO: 9 - DNA, 19 bp, artificial sequence
  forward primer LPFA1-CF1 for lpfA1 gene in E. coli ONTH26
ggttggtgacaaatccccg

SEQ ID NO: 10 - DNA, 20 bp, artificial sequence
  reverse primer LPFA1-CR1 for lpfA1 gene in E. coli ONTH26
gagaaccgtctggcctgttt

SEQ ID NO: 11 - DNA, 22 bp, artificial sequence
  forward primer LPFA1-B1F for lpfA1 gene in E. coli O113H21
ggtagtctggcgtcgccacaga

SEQ ID NO: 12 - DNA, 19 bp, artificial sequence
  reverse primer LPFA1-B1R for lpfA1 gene in E. coli O113H21
aatacgaataccaacgccg

SEQ ID NO: 13 - DNA, 21 bp, artificial sequence
  forward primer LPFA2-CF for lpfA2 gene in E. coli O157H7
ctacaggcggctgatggaaca

SEQ ID NO: 14 - DNA, 23 bp, artificial sequence
  reverse primer LPFA2-CR for lpfA2 gene in E. coli O157H7
gctaataccagcggcagcatcgt

SEQ ID NO: 15 - DNA, 22 bp, artificial sequence
  forward primer LPFA2-B2F for lpfA2 gene in E. coli O44
ggtagtctggcgtcaccacagc

SEQ ID NO: 16 - DNA, 19 bp, artificial sequence
  reverse primer LPFA2-B2F for lpfA2 gene in E. coli O44
aatacgaataccgacaccc